(19) United States
(12) Patent Application Publication
(10) Pub. No.: US 2020/0093804 A1
(43) Pub. Date: Mar. 26, 2020

(54) PHARMACEUTICAL COMPOSITION OF EVEROLIMUS

(71) Applicant: INTAS PHARMACEUTICALS LTD., Ahmedabad, Gujarat (IN)

(72) Inventors: Ravikumar Mahendrabhai ARDESHANA, Ahmedabad (IN); Sandip Kumar Manubhai PATEL, Ahmedabad (IN); Ashutosh JAMLOKI, Ahmedabad (IN); Ashish SEHGAL, Ahmedabad (IN)

(21) Appl. No.: 16/483,614

(22) PCT Filed: Feb. 5, 2018
(86) PCT No.: PCT/IB2018/050691

§ 371 Date: Aug. 5, 2019

(30) Foreign Application Priority Data
Feb. 6, 2017 (IN) 201721004195

(51) Int. Cl. A61K 31/436; A61K 9/20

(52) U.S. Cl. CPC A61K 31/436; A61K 9/2095; A61K 9/2054; A61K 9/2077

(57) ABSTRACT

The present invention relates to pharmaceutical composition comprising Everolimus and hot melt extrusion (HME) grade hydroxypropyl methylcellulose, and process for the preparation of the said composition.

### RELATED APPLICATIONS

This application is related to Indian Provisional Application 201721004195 filed 6 Feb. 2017 and is incorporated herein in its entirety.

### FIELD OF THE INVENTION

The present invention relates to pharmaceutical composition comprising Everolimus and hot melt extrusion (HME) grade hydroxypropyl methylcellulose, and process for the preparation of the said composition.

### BACKGROUND OF THE INVENTION

Everolimus is an inhibitor of mammalian target of rapamycin (mTOR), which is approved as an antineoplastic and immunosuppressant agent. The chemical name of Everolimus is (1R,9S,12S,15R,16E,18R,19R,21R,23S,24E,26E,28E,30S,32S,35R)-1,18dihydroxy-12-{(1R)-2-[(1S,3R,4R)-4-(2-hydroxyethoxy)-3-methoxy cyclohexyl]-1-methylethyl}-19,30-dimethoxy15,17,21,23,29,35-hexamethyl-11, 36-dioxa-4-aza-tricyclo[30.3.1.04,9]hexatriaconta-16,24,26,28-tetraene-2,3,10, 14,20pentaone. The molecular formula is C₅₃H₈₃NO₁₄ and the molecular weight is 958.2 and has following chemical structure:

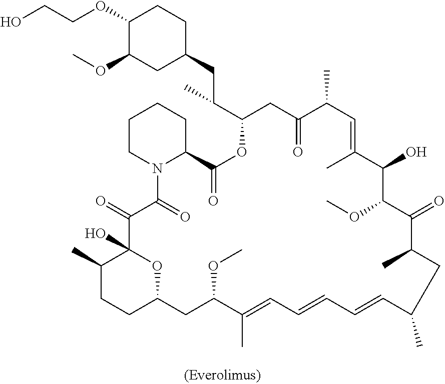 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

U.S. Pat. No. 5,665,772 discloses O-alkylated rapamycin derivatives which covers Everolimus. Everolimus is marketed as tablets (AFINITOR® and Zortress®), and tablets for oral suspension (AFINITOR DISPERZ®) by Novartis in USA. AFINITOR® is approved as anticancer agent and Zortress® is approved as immunosuppressant agent. AFINITOR® and Zortress® tablets contain anhydrous lactose, butylated hydroxytoluene, crospovidone, hypromellose, lactose monohydrate, and magnesium stearate as inactive ingredients. AFINITOR DISPERZ® contains butylated hydroxytoluene, colloidal silicon dioxide, crospovidone, hypromellose, lactose monohydrate, magnesium stearate, mannitol, and microcrystalline cellulose as inactive ingredients. Hypromellose used in the currently marketed composition is not melt extrusion (HME) grade.

From literature it is known that Everolimus has poor solubility and stability. To overcome the problem of solubility and stability several approaches are reported as follows:

U.S. Pat. No. 6,004,973 discloses compositions comprising solid dispersions in the form of co-precipitates, wherein solid dispersions contain rapamycin and a carrier medium, and methods of treatment utilizing such pharmaceutical compositions.

U.S. Pat. No. 7,297,703 discloses a mixture comprising a polyene macrolide and an antioxidant. The presence of the antioxidant improves the stability of the polyene macrolide to oxidation.

U.S. Pat. No. 7,741,338 discloses a solid mixture comprising 40-O-(2-hydroxy)ethyl-rapamycin and 2,6-di-tert-butyl-methylphenol (BHT).

U.S. Pat. No. 8,617,598 discloses a pharmaceutical composition comprising a macrolide solid dispersion, a disintegrant and colloidal silicon dioxide, wherein the composition comprises 1 to 5% colloidal silicon dioxide by weight.

Considering the prior efforts as disclosed in the background, a need exists which would address the issues relating to solubility of Everolimus in the pharmaceutical composition.

### OBJECT OF THE INVENTION

It is therefore, an object of the present invention is to provide pharmaceutical composition comprising Everolimus and hot melt extrusion (HME) grade hydroxypropyl methylcellulose.

Another object of the present invention is to provide process for the preparation of pharmaceutical composition comprising Everolimus and hot melt extrusion (HME) grade hydroxypropyl methylcellulose.

Another object of the present invention is to provide pharmaceutical composition comprising Everolimus and hot melt extrusion (HME) grade hydroxypropyl methylcellulose, wherein dissolution of Everolimus after 60 minutes from the composition of the present invention is faster than the composition using non HME grade hydroxypropyl methylcellulose when measured in USP apparatus II at 50 rpm using 500 ml purified water as dissolution media.

Another object of the present invention is to provide tablets comprising Everolimus and hot melt extrusion (HME) grade hydroxypropyl methylcellulose, wherein dissolution of Everolimus after 60 minutes from the composition of the present invention is faster than the composition using non HME grade hydroxypropyl methylcellulose when measured in USP apparatus II at 50 rpm using 500 ml purified water as dissolution media.

Another object of the present invention is to provide process for the preparation of pharmaceutical composition comprising Everolimus and hot melt extrusion (HME) grade hydroxypropyl methylcellulose, wherein dissolution of Everolimus after 60 minutes from the composition of the present invention is faster than the composition using non HME grade hydroxypropyl methylcellulose when measured in USP apparatus II at 50 rpm using 500 ml purified water as dissolution media.

### SUMMARY OF THE INVENTION

Present invention provides pharmaceutical composition comprising Everolimus and hot melt extrusion (HME) grade hydroxypropyl methylcellulose. Further the invention provides tablets comprising Everolimus and hot melt extrusion (HME) grade hydroxypropyl methylcellulose, wherein dissolution of Everolimus after 60 minutes from the composition of the present invention is faster than the composition using non HME grade hydroxypropyl methylcellulose when measured in USP apparatus II at 50 rpm using 500 ml purified water as dissolution media.

In another embodiment the present invention provides a process for preparation of pharmaceutical composition comprising Everolimus and hot melt extrusion (HME) grade hydroxypropyl methylcellulose.

### DETAILED DESCRIPTION OF THE INVENTION

Present invention provides pharmaceutical composition comprising Everolimus and hot melt extrusion (HME) grade hydroxypropyl methylcellulose.

In another embodiment, the present invention provides a process for the preparation of pharmaceutical composition comprising Everolimus and hot melt extrusion (HME) grade hydroxypropyl methylcellulose.

In another embodiment, the present invention provides pharmaceutical composition comprising Everolimus and hot melt extrusion (HME) grade hydroxypropyl methylcellulose, wherein dissolution of Everolimus after 60 minutes from the composition of the present invention is faster than the composition using non HME grade hydroxypropyl methylcellulose when measured in USP apparatus II at 50 rpm using 500 ml purified water as dissolution media.

Hot melt extrusion (HME) grade hydroxypropyl methylcellulose is currently marketed and available as AFFINISOL™ HPMC HME by Dow, which is designed for use in hot melt extrusion formulations. AFFINISOL™ HPMC HME is a water soluble amorphous polymer provided as a white to off-white powder currently available in 3 grades based on their molecular weight, i.e. HPMC HME 15 cps, HPMC HME 100 cps and HPMC HME 4M. The AFFINISOL™ HPMC HME has a glass transition temperature (Tg) of approximately 115° C. wherein non HME grade hypromellose typically exhibits a broad glass transition temperature from about 160° C. to 210° C.

Generally, it is known to the person skilled in the art that dissolution of drug from a pharmaceutical composition can be controlled by hydroxypropyl methylcellulose. As the viscosity of hydroxypropyl methylcellulose increases, the dissolution of drug from pharmaceutical composition decreases. The high viscosity grade hydroxypropyl methylcellulose releases drug slower than low viscosity grade and vice versa. For clarity, dissolution of drug from a composition having hydroxypropyl methylcellulose 15 cps (low viscosity grade) will be faster as compared to hydroxypropyl methylcellulose 100 cps (high viscosity grade). Surprisingly and contrary to the general knowledge of the person skilled in the art, the present invention provides pharmaceutical composition comprising Everolimus and hot melt extrusion (HME) grade hydroxypropyl methylcellulose 15 cps, wherein dissolution of Everolimus after 60 minutes from the composition of the present invention is faster than the composition using non HME grade hydroxypropyl methylcellulose 3 cps, when measured in USP apparatus II at 50 rpm using 500 ml purified water as dissolution media.

For the purpose of this specification, the term “non HME grade hypromellose” means hydroxypropyl methylcellulose which is not suitable for use in hot melt extrusion and has glass transition temperature is more than 160° C.

In one of the embodiment, the present invention provides a composition comprising Everolimus and hot melt extrusion (HME) grade hydroxypropyl methylcellulose.

In another embodiment, the present invention provides tablets comprising Everolimus and hot melt extrusion (HME) grade hydroxypropyl methylcellulose, wherein dissolution of Everolimus after 60 minutes from the composition of the present invention is faster than the composition using non HME grade hydroxypropyl methylcellulose when measured in USP apparatus II at 50 rpm using 500 ml purified water as dissolution media and process for preparation of same.

In another embodiment, the present invention provides pharmaceutical composition comprising Everolimus and hot melt extrusion (HME) grade hydroxypropyl methylcellulose, which further comprises of suitable excipients.

In another embodiment, the present invention provides tablets comprising Everolimus and hot melt extrusion (HME) grade hydroxypropyl methylcellulose, which further comprises of suitable excipients.

According to present invention suitable excipients may include, but not limited to diluent, disintegrant, lubricant, glidant, and like thereof.

According to present invention, diluent may include, but not limited to lactose anhydrous, lactose monohydrate, spray dried lactose, dicalcium phosphate, calcium phosphate tribasic, calcium carbonate, calcium sulfate, starch, corn starch, potato starch, wheat starch, pregelatinized starch, microcrystalline cellulose, silicified microcrystalline cellulose, cellulose microcrystalline powdered and the like, or mixtures thereof.

According to present invention, disintegrant may include, but not limited to crospovidone, starch, pregelatinized starch, sodium starch glycolate, ion-exchange resin and the like, or mixtures thereof.

According to present invention, lubricant may include, but not limited to calcium stearate, glyceryl behenate, magnesium stearate, mineral oil light, polyethylene glycol, castor oil, sodium stearyl fumarate, starch, stearic acid, talc, hydrogenated vegetable oil, zinc stearate, sodium benzoate and the like, or mixtures thereof.

According to present invention, glidant may include, but not limited to calcium silicate, magnesium silicate, colloidal silicon dioxide, talc and the like, or mixtures thereof.

In another embodiment, the present invention provides tablets comprising Everolimus, hot melt extrusion (HME) grade hydroxypropyl methylcellulose (AFFINISOL™ HPMC HME 15 cps), crospovidone, lactose anhydrous and magnesium stearate.

Another object of the present invention is to provide process for the preparation of pharmaceutical composition comprising Everolimus and hot melt extrusion (HME) grade hydroxypropyl methylcellulose, wherein dissolution of Everolimus after 60 minutes from the composition of the present invention is faster than the composition using non HME grade hydroxypropyl methylcellulose when measured in USP apparatus II at 50 rpm using 500 ml purified water as dissolution media.

In another embodiment, the present invention provides tablets comprising Everolimus, hot melt extrusion (HME) grade hydroxypropyl methylcellulose (AFFINISOL™ HPMC HME 15 cps), crospovidone, lactose anhydrous and magnesium stearate, wherein dissolution of Everolimus after 60 minutes from the composition is faster than the composition using non HME grade hydroxypropyl methylcellulose when measured in USP apparatus II at 50 rpm using 500 ml purified water as dissolution media.

In another embodiment, the present invention provides tablets comprising Everolimus, hot melt extrusion (HME) grade hydroxypropyl methylcellulose (AFFINISOL™ HPMC HME 15 cps), crospovidone, lactose anhydrous and magnesium stearate, wherein the tablet is prepared by a process comprising granulation step.

In another embodiment, the present invention provides tablets comprising Everolimus, hot melt extrusion (HME) grade hydroxypropyl methylcellulose (AFFINISOL™ HPMC HME 15 cps), crospovidone, lactose anhydrous and magnesium stearate, wherein the tablet is prepared by a process comprising granulation step, and wherein the solvent used as granulating fluid is mixture of isopropyl alcohol and dichloromethane.

In another embodiment, the present invention provides a process for the preparation of pharmaceutical composition comprising Everolimus and hot melt extrusion (HME) grade hydroxypropyl methylcellulose comprising step of:
1. Sifting the hot melt extrusion grade hydroxypropyl methylcellulose and suitable excipients through suitable sieve,
2. Mixing the suitable excipients and hot melt extrusion grade hydroxypropyl methylcellulose,
3. Dissolving Everolimus and optionally binder in a suitable solvent,
4. Granulating the material obtained in step 2 using solution obtained in step 3,
5. Drying the granulated mixture of step 4 using a suitable dryer,
6. Sifting the suitable extra-granular excipients,
7. Mixing the dried granules obtained in step 5 with extra-granular excipients obtained in step 6 to obtain a blend,
8. Lubricating the blend using suitable lubricant, and
9. Compressing the lubricated blend using a compression machine to obtain suitable tablets.

In another embodiment, the present invention provides a process for the preparation of pharmaceutical composition comprising Everolimus and hot melt extrusion (HME) grade hydroxypropyl methylcellulose comprising step of:
1. Granules were prepared comprising Lactose anhydrous, AFFINISOL™ HPMC HME 15, and Everolimus.
2. Crospovidone and lactose anhydrous and magnesium stearate were added to the granules.
3. The lubricated granules were compressed into tablets,

wherein, Granules are prepared using rapid mixer granulator (RMG).

In another embodiment, the present invention provides a process for the preparation of pharmaceutical composition comprising Everolimus and hot melt extrusion (HME) grade hydroxypropyl methylcellulose comprising step of:
1. Granules were prepared comprising Lactose anhydrous, AFFINISOL™ HPMC HME 15, and Everolimus.
2. Crospovidone and lactose anhydrous and magnesium stearate were added to the granules.
3. The lubricated granules were compressed into tablets.

wherein, Granules are prepared using fluid bed granulator (FBG).

### EXAMPLES

The present invention has been described by way of example only, and it is to be recognized that modifications thereto falling within the scope and spirit of appended claims, and which would be obvious to a person skilled in the art based upon the disclosure herein, are also considered to be within the scope of this invention.

### Example 1: Everolimus Tablets 10 mg Composition

Everolimus tablets 10 mg composition

| Composition details | | Batch using RMG | | Batch using FBG | |
|---|---|---|---|---|---|
| Sr. No. | Ingredients | mg/Tablet | % w/w | mg/Tablet | % w/w |
| Intra granular ingredients | | | | | |
| 1 | Everolimus | 10 | 2 | 10 | 2 |
| 2 | Lactose anhydrous | 207.5 | 41.5 | 207.5 | 41.5 |
| 4 | AFFINISOL ™ | 90 | 18 | 90 | 18 |
| | HPMC HME 15 cps | | | | |
| 5 | Isopropyl alcohol^{#} | q.s. | — | q.s. | — |
| 6 | Dichloromethane^{#} | q.s. | — | q.s. | — |
| Extra granular ingredients | | | | | |
| 7 | Crospovidone | 100 | 20 | 100 | 20 |
| 8 | Lactose anhydrous | 90 | 18 | 90 | 18 |
| 9 | Magnesium stearate | 2.5 | 0.5 | 2.5 | 0.5 |
| TOTAL | | 500 | 100 | 500 | 100 |

^{#}Not present in final composition

Manufacturing process of example 1:
1. Granules were prepared comprising Lactose anhydrous, AFFINISOL™ HPMC HME 15, and Everolimus.
2. Crospovidone and lactose anhydrous and magnesium stearate were added to the granules.
3. The lubricated granules were compressed into tablets.

The granules were prepared using Rapid Mixer Granulator (RMG) or Fluid Bed Granulator (FBG).

### Comparative Example 1: Everolimus Tablets 10 mg Composition

Everolimus tablets 10 mg composition

| Composition details | | Batch using RMG | | Batch using FBG | |
|---|---|---|---|---|---|
| Sr. No. | Ingredients | mg/Tablet | % w/w | mg/Tablet | % w/w |
| Intra granular Ingredients | | | | | |
| 1 | Everolimus | 10 | 2 | 10 | 2 |
| 2 | Lactose anhydrous | 297.5 | 59.5 | 207.5 | 41.5 |
| 4 | Hypromellose 3 cps | 90 | 18 | 90 | 18 |
| 5 | Isopropyl alcohol^{#} | q.s. | — | q.s. | — |
| 6 | Dichloromethane^{#} | q.s. | — | q.s. | — |
| Extra granular Ingredients | | | | | |
| 7 | Crospovidone | 100 | 20 | 100 | 20 |
| 8 | Lactose anhydrous | — | — | 90 | 18 |
| 9 | Magnesium stearate | 2.5 | 0.5 | 2.5 | 0.5 |
| TOTAL | | 500 | 100 | 500 | 100 |

^{#}Not present in final composition

Manufacturing process: The comparative example 1 was prepared according to process similar as of example 1.

### Following are the Dissolution Study Results of the Tablets Obtained According to Example 1 and Comparative Example 1.

### Dissolution Study Results:

The dissolution study of the tablets prepared according to the invention (i.e. examples 1) and comparative example 1 were carried out in USP apparatus II at 50 RPM using 500 ml purified water as dissolution media at 37° C. The obtained dissolution study results are tabulated below.

| | | Tablets of | Tablets of |
|---|---|---|---|
| Apparatus: | USP apparatus | example 1 | comparative |
| Media: | II @ 50 RPM | prepared using | example 1 |
| Volume: | Purified water | AFFINISOL ™ | prepared using |
| Dissolution | 500 mL | HPMC HME 15 | Hypromellose 3 |
| Data | Time in minutes | cps in FBG | cps in FBG |
| | 5 | 54% | 31% |
| | 10 | 70% | 39% |
| | 15 | 78% | 45% |
| | 20 | 81% | 49% |
| | 30 | 85% | 55% |
| | 45 | 86% | 59% |
| | 60 | 86% | 60% |

| | |
|---|---|
| Conclusion | Dissolution of Everolimus after 60 minutes from tablet of |
| | example 1 is faster than the tablet of comparative example 1 |

Thus, pharmaceutical composition comprising Everolimus and hot melt extrusion (HME) grade hydroxypropyl methylcellulose can be prepared according to the present invention, wherein dissolution of Everolimus after 60 minutes from the composition according to present invention is faster than the composition using non HME grade hydroxypropyl methylcellulose. The pharmaceutical composition according to the present invention would be useful in achieving bioequivalence with regards to the reference drug product.

1. A pharmaceutical composition comprising Everolimus and hot melt extrusion grade hydroxypropyl methylcellulose.

2. The pharmaceutical composition according to claim 1, wherein hot melt extrusion grade hydroxypropyl methylcellulose has a glass transition temperature (Tg) of approximately 115° C.

3. The pharmaceutical composition according to claim 2, wherein dissolution of Everolimus after 60 minutes from the composition is faster than the composition using non HIE grade hydroxypropyl methylcellulose; when measured in USP apparatus II at 50 rpm using 500 ml purified water as dissolution media.

4. The pharmaceutical composition according to claim 1, which further comprises suitable excipients.

5. The pharmaceutical composition according to claim 4, wherein the suitable excipients comprises of diluent, disintegrant and lubricant.

6. The pharmaceutical composition according to claim 1 is a tablet.

7. The pharmaceutical composition according to claim 6, wherein the tablet is prepared by a process comprising granulation step.

8. A pharmaceutical composition comprising Everolimus and hot melt extrusion grade hydroxypropyl methylcellulose, wherein dissolution of Everolimus after 60 minutes from the composition is faster than the composition using non HME grade hydroxypropyl methylcellulose when
measured in USP apparatus II at 50 rpm using 500 ml purified water as dissolution media.
